(19) United States
(12) Patent Application Publication
(10) Pub. No.: US 2004/0014994 A1
(43) Pub. Date: Jan. 22, 2004

(54) Method for resolving chiral (2s) and (2r) chromanes

(76) Inventors: Luc Antoine, Landen (BE); Pascal Bouquel, Montmedy (FR); Alfio Borghese, Dreve M. Davout Baisy-Thy (IT); Hugo Gorissen, Avenue Felix Lacourt Grez-Dioceau (BE); Vien V. Khau, Shafer Court Carmel, IN (US); Michael Martinelli, Mulsanne Drive Zionsville, IN (US); Alain Merschaert, Rue Fontaine a Louche Petit-Ehghien (BE); Gerd Ruhter, Vierzigstuecken Hamburg (DE); Carine Rypens, 2,allee des Rouges-Gorges Palaiseau (FR)

Correspondence Address: KNOBBE MARTENS OLSON & BEAR LLP, 2040 MAIN STREET, FOURTEENTH FLOOR, IRVINE, CA, 92614, US

(21) Appl. No.: 10/296,538

(22) PCT Filed: Jun. 1, 2001
(86) PCT No.: PCT/US01/17977

(51) Int. Cl. C07D 311/58

(52) U.S. Cl. 549/404

(57) ABSTRACT

Disclosed are processes for resolving chiral (2S) and (2R) benzopyrans, racemizing benzopyrans, and recycling racemized benzopyrans to increase yield of a desired enantiomer to provide purified or substantially purified bicyclic amino substituted benzopyran derivatives. Such benzopyran derivatives are preferably chromans which can be coupled with benzoyl derivatives via an amide bond to produce potent platelet aggregation inhibitors.

### FIELD OF THE INVENTION

This invention relates to processes for resolving chiral (2S) and (2R) chromanes, racemizing chromanes, and recycling racemized chromanes to increase yield of a desired enantiomer to provide purified or substantially purified bicyclic amino substituted benzopyran derivatives. Such benzopyran derivatives are preferably chromans which can be coupled with benzoyl derivatives via an amide bond to produce therapeutic agents, or where the compounds themselves are therapeutic agents, for disease states in mammals that have disorders caused by or impacted by platelet dependent narrowing of the blood supply.

### BACKGROUND OF THE INVENTION

One process for producing amino-substituted chromanes for coupling with benzoyl derivatives is described in U.S. Pat. No. 5,731,324 at pages 147 and 148 (Examples E and F). However, that process of coupling a carbonyl nitrile derivative to the amino-substituted benzopyran produces an racemate and has an overall yield from coupling and conversion of the cyano group which is rather low. If the bicyclic starting material is a resolved single enantiomer, the loss of 64% of the enantiomer in the coupling step is very expensive. Thus, there is need for an efficient and lower-cost way for producing and resolving single enantiomers as well as processes for recycling the other enantiomer of the racemate. The carbonyl nitrile derivative may be substituted by various groups as described in U.S. Pat. No. 5,731,324, such as halogen: see for example, Scheme 17 on pages 77-78, wherein 2-fluoro-4-nitrile benzoic acid is coupled with a tetralone compound. Also, the bicyclic ring may be substituted in other positions and still have the need to resolve the (2) position chiral carbon intermediates into a single enantiomer for coupling.

### SUMMARY OF THE INVENTION

Accordingly, there is a need for improved processes for producing chiral intermediates of bicyclic compounds such as benzopyrans substituted by an amino group or a protected amino group and carbonyl derivatives (or salts thereof) which are useful as intermediates for coupling with a carbonyl group to produce a carboxamide link to result in compounds that are useful platelet aggregation inhibitors or intermediates for forming platelet aggregation inhibitors. Also needed is a process to produce, in a relatively inexpensive manner, large quantities of such intermediates that are useful for producing substantially pure compositions of a single enantiomer (R or S enantiomer) of the platelet aggregation inhibitor compounds. One or more of the foregoing needs may be met using the processes described herein and the compounds and intermediates made thereby.

The present invention relates to novel processes for producing enriched enantiomeric compositions of benzopyrans, preferably chromans, which are used to produce therapeutic agents, or where the compounds themselves are therapeutic agents, for disease states in mammals that have disorders caused by or impacted by platelet dependent narrowing of the blood supply.

In accordance with one preferred embodiment, there is provided a process for making enantiomerically enriched 2-[(S>R) 6-aminochroman-2-yl] acetic acid. The process comprises (a) through (g) below as process steps:

(a) protecting the amino group by conversion to an acetamido group as follows:

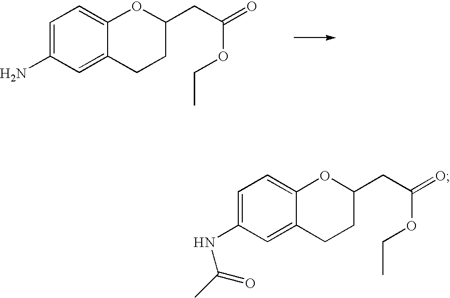

(b) converting the ester to the free acid;

(c) reacting the free acid with a slight excess of D-alaninol in alcholic solution, and separating a first quantity of diastereomeric salt as follows:

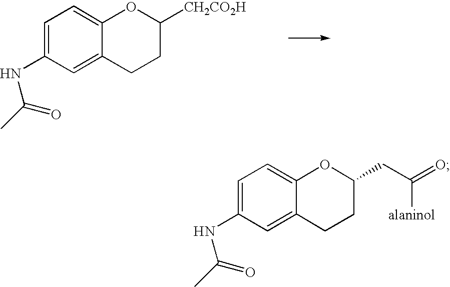

(d) adding thionyl chloride to the (R>S) mixture in the mother liquor containing ROH to esterify the acid followed by neutralization as follows:

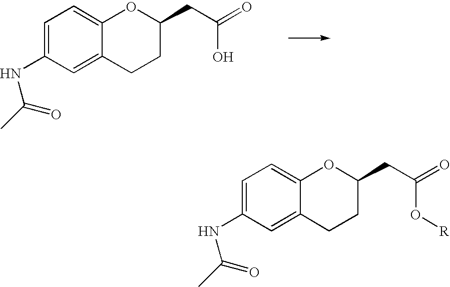

where R is C₁-C₆ alkyl;

(e) racemizing the compound from the mother liquor at the 2-position by opening and closing the pyran ring by the addition of a base:

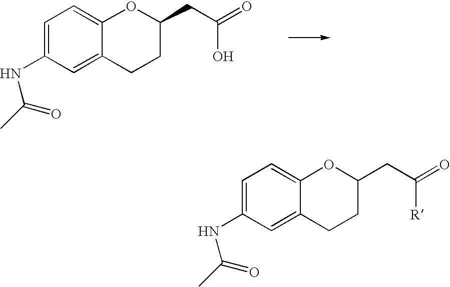

where R′ is H or C₁-C₆ alkyl;

(f) repeating the procedure of (c), either directly of after repeating the procedure of (b), with the solution obtained in (e) to obtain a second quantity of diastereomeric salt, wherein the procedure of (b) is repeated if R′ is not H; and

(g) heating the first and second quantities of diastereomeric salt in a solvent comprising ROH in the presence of acid followed by neutralization as follows:

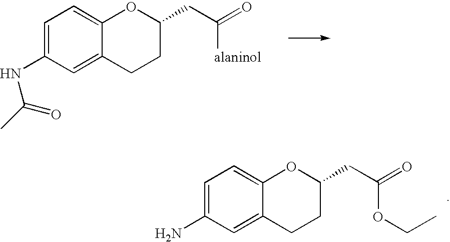

In a preferred embodiment, the process further comprises addition of an acid halide to an organic solution of the product of (g) followed by recovery of the precipitated amino halide salt.

In accordance with another preferred embodiment, there is provided a process for making enantiomerically enriched 2-[(S>R) 6-aminochroman-2-yl] acetic acid. The process comprises reaction steps (a) through (h) as follows:

(a) protecting the amino group by conversion to an acetamido group as follows:

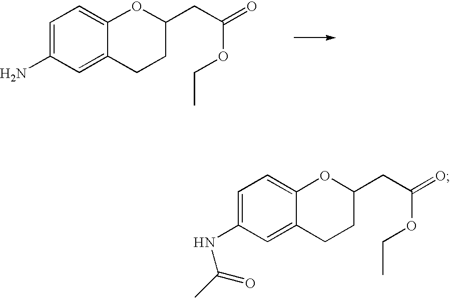

(b) converting the ester to the free acid;

(c) reacting the free acid with a slight excess of D-alaninol in alcholic solution, and separating a first quantity of diastereomeric salt as follows:

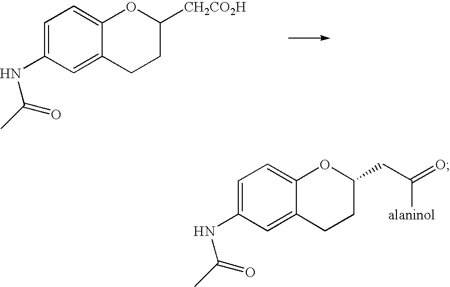

(d) adding potassium or sodium hydroxide to the (R)>(S) mother liquor, followed by acid to precipitate the 2-[6-acetamido-chroman-2-yl] acetic acid;

(e) dissolving the product from (d) in solvent comprising ROH and adding thionyl chloride to esterify the acid as follows:

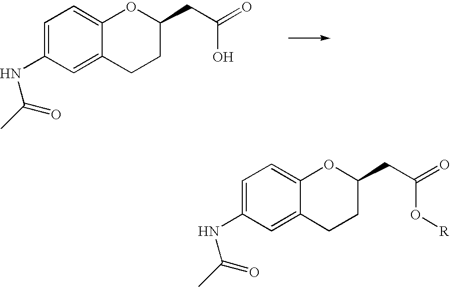

where R is C₁-C₆ alkyl;

(f) racemizing the compound in the mother liquor at the 2-position by opening and closing the pyran ring by the addition of a base:

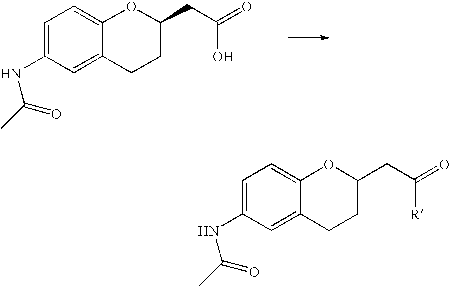

where R′ is H or C₁-C₆ alkyl;

(g) repeating the procedure of (c), either directly of after repeating the procedure of (b), with the solution obtained in (f) to obtain a second quantity of diastereomeric salt, wherein the procedure of (b) is repeated if R′ is not H; and

(h) heating the first and second quantities of diastereomeric salt in a solvent comprising ROH in the presence of acid followed by neutralization as follows:

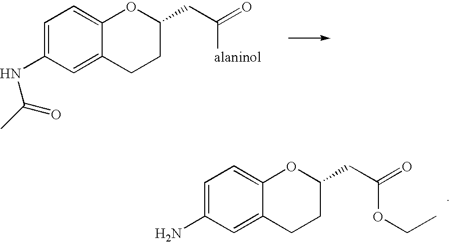

In a preferred embodiment, the process further comprises addition of an acid halide to an organic solution of the product of (h) followed by recovery of the precipitated amino halide salt.

### DETAILED DESCRIPTION OF THE PREFERRED EMBODIMENTS

Processes for Producing The Bicylic Ring Portion for Coupling

For the purposes of the disclosure herein, any amino substituted bicyclic (2S/2R) benzopyran (or benzothiopyran) compound useful for making platelet aggregation inhibitor compounds may be resolved into the (2S) or (2R) compounds for a coupling reaction step. Examples of such bicyclic compounds can be found at pages 5-6 of U.S. Pat. No. 5,731,324, the entire disclosure of which is hereby incorporated by reference. For exemplification purposes, in the paragraph bridging pages 147 and 148 of that patent is described a racemic (2S/2R) bicyclic free amino-substituted acetic acid ester compound (described as a dark oil) having the following formula:

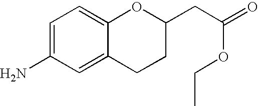

If desired, one or both of the amino group and the ester group of the acetic acid side chain can be modified with a group which can be utilized to resolve the R or S enantiomers. For example, the ester group can be reacted with a conventional camphor sulphonic acid derivative, a dibenzoyl tartaric acid derivative, (S) or (D) alaninol, and the like, to produce a desired enantiomer. The amino group can be protected by converting the it to an acetamido group, as follows:

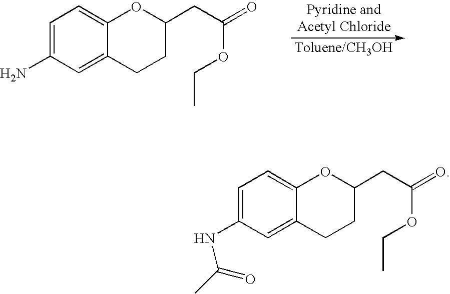

In a preferred embodiment, the racemic ethyl 2-[6-acetamidochroman-2-yl]acetate is converted to the free acid by reaction with a base such as sodium hydroxide and neutralized with an acid such as hydrochloric acid. Preferably, the free acid is then reacted with a slight excess of ½ mole of (S) or (D) alaninol per mole of racemate in the presence of methanol. One of the enantiomers will preferentially react with the alaninol and crystallize out (depending upon whether (S) or (D) alaninol is used as the reactant). The crystalline salt can be rinsed by an appropriate solvent such as isopropyl alcohol and may be further purified by recrystallization in an appropriate solvent such as methanol or a mixture of isopropanol and ethanol, and the like. This procedures and the optional recrystallization result in an enriched, essentially pure, or pure crystalline composition of the alaninol salt of either the (R) or (S) enantiomer.

Both the alaninol and the amino protecting group can be removed from the crystalline alaninol salt by heating the salt in an appropriate solvent in the presence of an acid. Preferably, the acid is sulfuric acid and the solvent is absolute ethanol. Upon concentration of the reaction mixture by evaporation of the solvent, the mixture is neutralized with an appropriate base (such as sodium hydrogen carbonate and the like) in the presence of an appropriate solvent (such as toluene and the like). The aqueous layer can be separated from the organic layer and the aqueous layer extracted with toluene. After pooling of the organic layers, an acid halide can be added to precipitate the amino halide salt from the toluene solvent as follows:

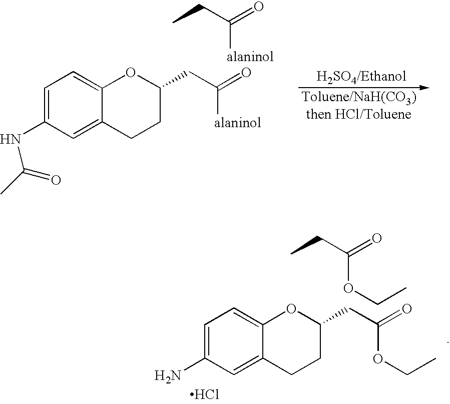

The halide salt can optionally be recrystallized in an appropriate solvent such ethanol, ethyl acetate, ethanol/isopropyl alcohol, and the like to produce a higher enantiomeric purity. Preferably, the crude enantiomer is heated in absolute alcohol or denatured absolute alcohol (no methanol) or the like, and recrystallized. If the preferred enantiomer remains in the solvent, the solvent can be evaporated to yield the purer form of the single enantiomer halide salt.

In the above description, an alcoholic sulfuric acid solution followed by an alcoholic HCI solution are utilized for the esterification and to cause the amino group to form a hydrochloride salt, but other esters such as the methyl or propyl ester may likewise be used. The purity of the enantiomer my be optionally improved by recrystallization, HPLC or the like. The preferred solvent for recrystallization is methanol or isopropyl alcohol or a mixture thereof. In either event, after resolving the desired (2S or 2R)-enantiomer and obtaining the 2-carboxylic acid form of the molecule, the acidic alcoholic solutions may be added to the free acid R or S enantiomer compound to form the ester of the 2-carboxylic acid and an excess of HCl is then utilized to produce the hydrochloride salt of the amino group.

The amino halide salt of the bicyclic compound thus produced can be utilized in a coupling reaction with a carbonyl compound or a halide salt of a carbonyl compound to provide a carboxamide coupled compound, as described in greater detail below.

Processes for Recycling an (2R) or (2S) Enantiomer

In the above chiral resolution process there may be produced a desired or undesired (2S) or (2R) enantiomer alaninol salt. To increase the yield of a single enantiomer, it is advantageous to recycle the free acid (2S) or (2R) 6-acetamido bicyclic enantiomer still dissolved in an alcohol (methanol or ethanol) from the alaninol salt precipitation step which did not react to form an alaninol salt. This can be done by opening and closing the pyran ring with a base such as metal alcoholate and the like, such as sodium methoxide, sodium ethoxide, sodium isopropoxide, sodium t-butylate, potassium methoxide, potassium ethoxide, potassium isopropoxide, potassium t-butylate, LHMDA, sodium hydride, potassium hydride and the like. The ideal basic alcoholate for the racemization corresponds to the alcohol that will be used to esterify the acidic side chain to avoid esterification of the acidic side chain during the racemization which could lead to mixed ester impurities that could be difficult to separate. In the instant example for the ethyl ester, a metallic ethoxide such as sodium ethoxide or potassium oxide is preferred in a solvent that will also avoid the formation of an undesired ester, for example ethyl acetate or ethanol. The process can be illustrated using potassium methoxide as follows, but use of ethanol for esterification and the solvent along with potassium ethoxide as the base are preferred:

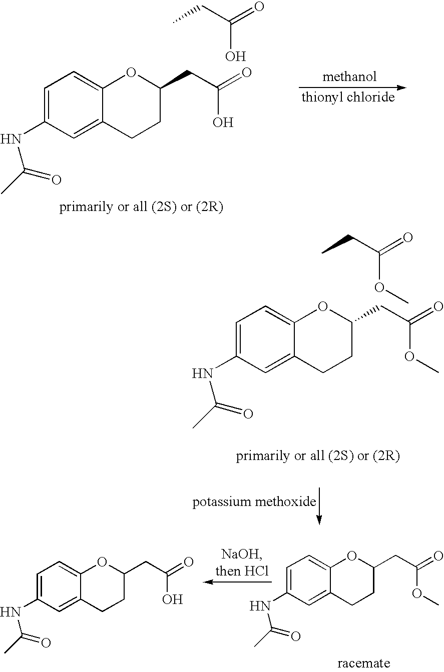

Processes for Producing the Carbonyl Substituted Ring Portion for Coupling

For the purposes of the disclosure herein, any basic substituted carbonyl group useful for making platelet aggregation inhibitor compounds may be utilized for the coupling reaction step. Examples of suitable carbonyl derivative compounds are incorporated by reference to U.S. Pat. No. 5,731,324, particularly at pages 17-20, with the substituted or unsubstituted amidino-benzoyl or an amidino thiophenoyl derivatives being especially preferred. Even more preferably, the carbonyl derivative is an amidinobenzoyl derivative, which is optionally substituted by a halogen atom. And even more preferably, is a member selected from the group consisting of 4-amidinobenzoic acid, 4-amidino-2-fluorobenzoicacid, 4-amidino-2-bromobenzoic acid, 4-amidino-2-iodobenzoic acid and 4-amidino-2-chlorobenzoic acid. For exemplification purposes, on page 20, at formula IV, is shown coupled 4-amidino-2-fluorobenzoic acid, which is readily obtained from the nitrile shown in Scheme 17, pages 77-78, by converting the cyano group to a amidino group using procedures described in the U.S. Pat. No. 5,731,324. Preferably, the acid halide for coupling is 4-cyano-2-fluorobenzoyl chloride or 4-cyanobenzoyl chloride.

Processes for Coupling the Amine Salt and the Acyl Halide Salt to Form a Carboxamide

The coupling process as shown in the Example 47 E and F of U.S. Pat. No. 5,731,324 may be utilized for producing a single enantiomer from the (2R) or (2S) bicyclic amine enantiomer obtainable by separation with a resolving agent such as alaninol as described above. For the purposes of the disclosure herein any salt of an aminobicyclic compound and any basic substituted carbonyl group that are useful for coupling to make platelet aggregation inhibitor compounds may be utilized for the coupling reaction step. However, for purposes of illustration only, the above described ethyl (R or S) (6-aminochroman-2-yl)acetate hydrochloride and 4-amidino-2-fluorobenzoyl chloride, will be utilized.

The (S) and (R) enantiomers of ethyl 2-(6-(4-amidino-2-fluorobenzoyl)amino-chroman-2-yl)acetate (and other compounds produced by the above methods but utilizing the different carbonyl and bicyclic structures set forth in U.S. Pat. No. 5,731,324) may be used as potent therapeutic agents therapeutic agents for disease states in mammals which have disorders that are due to platelet dependent narrowing of the blood supply, such as atherosclerosis and arteriosclerosis, acute myocardial infarction, chronic stable angina, unstable angina, transient ischemic attacks and strokes, peripheral vascular disease, arterial thrombosis, preclampsia, embolism, restenosis following angioplasty, carotid endarterectomy, anastomosis of vascular grafts, and etc. These conditions represent a variety of disorders thought to be initiated by platelet activation on vessel walls.

Therefore, one embodiment is directed to such (S) and (R) enantiomers in pure, substantially pure or enriched form as therapeutic agents for treating such disorders, and pharmaceutical compositions comprising an effective amount of such compounds.

In another embodiment, there is a method comprising administering to a patient in need thereof a pharmaceutical composition comprising an effective amount of either the (S) or (R) enantiomer of ethyl 2-(6-(4-amidino-2-fluorobenzoyl)amino-chroman-2-yl)acetate, the free acid, or other esters and salts thereof.

Uses of Compounds

As mentioned above, the compounds disclosed herein find utility as intermediates for producing therapeutic agents or as therapeutic agents for disease states in mammals which have disorders that are due to platelet dependent narrowing of the blood supply, such as atherosclerosis and arteriosclerosis, acute myocardial infarction, chronic stable angina, unstable angina, transient ischemic attacks and strokes, peripheral vascular disease, arterial thrombosis, preclampsia, embolism, restenosis following angioplasty, carotid endarterectomy, anastomosis of vascular grafts, and etc. These conditions represent a variety of disorders thought to be initiated by platelet activation on vessel walls.

Platelet adhesion and aggregation is believed to be an important part of thrombus formation. This activity is mediated by a number of platelet adhesive glycoproteins. The binding sites for fibrinogen, fibronectin and other clotting factors have been located on the platelet membrane glycoprotein complex IIb/IIIa. When a platelet is activated by an agonist such as thrombin the GPIIb/IIIa binding site becomes available to fibrinogen, eventually resulting in platelet aggregation and clot formation. Thus, intermediate compounds for producing compounds that effective in the inhibition of platelet aggregation and reduction of the incidence of clot formation are useful intermediate compounds.

The compounds produced according to the methods disclosed herein may used as intermediates for producing therapeutic compounds or as compounds that may be administered in combination or in concert with other therapeutic or diagnostic agents. In certain preferred embodiments, the compounds produced by the intermediates according to the disclosure herein may be co-administered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds produced from the intermediates according to preferred embodiments may act in a synergistic fashion to prevent reocclusion following a successful thrombolytic therapy and/or reduce the time to reperfusion. Such compounds may also allow for reduced doses of the thrombolytic agents to be used and therefore minimize potential hemorrhagic side-effects. Such compounds can be utilized in vivo, ordinarily in mammals such as primates, (e.g. humans), sheep, horses, cattle, pigs, dogs, cats, rats and mice, or in vitro.

The starting materials used in above processes are commercially available from chemical vendors such as Aldrich, Sigma, Nova Biochemicals, Bachem Biosciences, and the like, or may be readily synthesized by known procedures, for example, by using procedures such as indicated above.

Reactions are carried out in standard laboratory glassware and reaction vessels under reaction conditions of standard temperature and pressure, except where otherwise indicated, or is well-known in literature available in the art. Further, the above procedures of the processes described herein may be carried out on a commercial scale by utilizing reactors and standard scale-up equipment available in the art for producing large amounts of compounds in the commercial environment. Such equipment and scale-up procedures are well-known to the ordinary practitioner in the field of commercial chemical production.

During the synthesis of these compounds, amino or acid functional groups may be protected by blocking groups to prevent undesired reactions with the amino group during certain procedures. Examples of suitable blocking groups are well known in the art. Further, removal of amino or acid blocking groups by procedures such as acidification or hydrogenation are well-known in the art.

Compositions and Formulations

The compounds according to preferred embodiments may be isolated as the free acid or base or converted to salts of various inorganic and organic acids and bases. Such salts are within the scope of this disclosure and are presently contemplated. Non-toxic and physiologically compatible salts are particularly useful although other less desirable salts may have use in the processes of isolation and purification.

A number of methods are useful for the preparation of the salts described above and are known to those skilled in the art. For example, reaction of the free acid or free base form of a compound of the structures recited above with one or more molar equivalents of the desired acid or base in a solvent or solvent mixture in which the salt is insoluble, or in a solvent like water after which the solvent is removed by evaporation, distillation or freeze drying. Alternatively, the free acid or base form of the product may be passed over an ion exchange resin to form the desired salt or one salt form of the product may be converted to another using the same general process.

Diagnostic applications of compounds according to preferred embodiments disclosed herein will typically utilize formulations such as solution or suspension. In the management of thrombotic disorders preferred compounds according to the present disclosure may be utilized in compositions such as tablets, capsules or elixirs for oral administration, suppositories, sterile solutions or suspensions for injectable or parenteral administration, and the like, or incorporated into shaped articles. Subjects in need of treatment (typically mammalian) using the compounds according to the present disclosure can be administered dosages that will provide optimal efficacy. The dose and method of administration will vary from subject to subject and be dependent upon such factors as the type of mammal being treated, its sex, weight, diet, concurrent medication, overall clinical condition, the particular compounds employed, the specific use for which these compounds are employed, and other factors which those skilled in the medical arts will recognize.

Formulations of the compounds disclosed herein are prepared for storage or administration by mixing the compound, or a pharmaceutically acceptable salt, solvate or prodrug thereof, having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counter ions such as sodium and/or nonionic surfactants such as Tween, Pluronics or polyethyleneglycol.

Dosage formulations of the present compounds to be used for parenteral administration are preferably sterile. Sterility is readily accomplished by filtration through sterile membranes such as 0.2 micron membranes, or by other conventional methods known to those skilled in the art. Formulations are preferably stored in lyophilized form or as an aqueous solution. The pH of the preparations are preferably between 3 and 11, more preferably from 5 to 9 and most preferably from 7 to 8. It will be understood that use of certain of the foregoing excipients, carriers, or stabilizers will result in the formation of cyclic polypeptide salts. While the preferred route of administration is by injection, other methods of administration are also anticipated such as intravenously (bolus and/or infusion), subcutaneously, intramuscularly, colonically, rectally, nasally or intraperitoneally, employing a variety of dosage forms such as suppositories, implanted pellets or small cylinders, aerosols, oral dosage formulations and topical formulations such as ointments, drops and dermal patches. The compounds according to the present disclosure are desirably incorporated into shaped articles such as implants which may employ inert materials such as biodegradable polymers or synthetic silicones, for example, Silastic, silicone rubber or other polymers commercially available.

The compounds according to the present disclosure may also be administered in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The compounds according to the present disclosure may also be delivered by the use of antibodies, antibody fragments, growth factors, hormones, or other targeting moieties, to which the compound molecules are coupled. The compounds may also be coupled with suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the platelet aggregation inhibitors disclosed herein may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

Therapeutic compound liquid formulations generally are placed into a container having a sterile access port, for example, an intravenous solution bag or vial having a stopper pierceable by hypodermic injection needle.

Therapeutically effective dosages may be determined by either in vitro or in vivo methods. For each particular compound presently disclosed, individual determinations may be made to determine the optimal dosage required. The range of therapeutically effective dosages will naturally be influenced by the route of administration, the therapeutic objectives, and the condition of the patient. For injection by hypodermic needle, it may be assumed the dosage is delivered into the body's fluids. For other routes of administration, the absorption efficiency must be individually determined for each inhibitor by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be within the ambit of one skilled in the art. Typically, applications of compound are commenced at lower dosage levels, with dosage levels being increased until the desired effect is achieved.

A typical dosage might range from about 0.001 mg/kg to about 1000 mg/kg, preferably from about 0.01 mg/kg to about 100 mg/kg, and more preferably from about 0.10 mg/kg to about 20 mg/kg. Advantageously, the compounds disclosed herein may be administered several times daily, and other dosage regimens may also be useful.

Typically, about 0.5 to 500 mg of a compound or mixture of compounds, as the free acid or base form or as a pharmaceutically acceptable salt, is compounded with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained.

Typical adjuvants which may be incorporated into tablets, capsules and the like are a binder such as acacia, corn starch or gelatin, and excipient such as microcrystalline cellulose, a disintegrating agent like corn starch or alginic acid, a lubricant such as magnesium stearate, a sweetening agent such as sucrose or lactose, or a flavoring agent. When a dosage form is a capsule, in addition to the above materials it may also contain a liquid carrier such as water, saline, a fatty oil. Other materials of various types may be used as coatings or as modifiers of the physical form of the dosage unit. Sterile compositions for injection can be formulated according to conventional pharmaceutical practice. For example, dissolution or suspension of the active compound in a vehicle such as an oil or a synthetic fatty vehicle like ethyl oleate, or into a liposome may be desired. Buffers, preservatives, antioxidants and the like can be incorporated according to accepted pharmaceutical practice.

In practicing the methods disclosed herein, the compounds may be used alone or in combination, or in combination with other therapeutic or diagnostic agents. In certain preferred embodiments, the compounds disclosed herein may be coadministered along with other compounds typically prescribed for these conditions according to generally accepted medical practice, such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds disclosed herein can be utilized in vivo, ordinarily in mammals such as primates, such as humans, sheep, horses, cattle, pigs, dogs, cats, rats and mice, or in vitro.

The preferred compounds disclosed herein are characterized by their ability to inhibit thrombus formation with acceptable effects on classical measures of coagulation parameters, platelets and platelet function, and acceptable levels of bleeding complications associated with their use. Conditions characterized by undesired thrombosis would include those involving the arterial and venous vasculature.

With respect to the coronary arterial vasculature, abnormal thrombus formation characterizes the rupture of an established atherosclerotic plaque which is the major cause of acute myocardial infarction and unstable angina, as well as also characterizing the occlusive coronary thrombus formation resulting from either thrombolytic therapy or percutaneous transluminal coronary angioplasty (PTCA).

With respect to the venous vasculature, abnormal thrombus formation characterizes the condition observed in patients undergoing major surgery in the lower extremities or the abdominal area who often suffer from thrombus formation in the venous vasculature resulting in reduced blood flow to the affected extremity and a predisposition to pulmonary embolism. Abnormal thrombus formation further characterizes disseminated intravascular coagulopathy commonly occurs within both vascular systems during septic shock, certain viral infections and cancer, a condition wherein there is rapid consumption of coagulation factors and systemic coagulation which results in the formation of life-threatening thrombi occurring throughout the microvasculature leading to widespread organ failure.

The compounds disclosed herein, selected and used as disclosed herein, are believed to be useful for preventing or treating a condition characterized by undesired thrombosis, such as (a) the treatment or prevention of any thrombotically mediated acute coronary syndrome including myocardial infarction, unstable angina, refractory angina, occlusive coronary thrombus occurring post-thrombolytic therapy or post-coronary angioplasty, (b) the treatment or prevention of any thrombotically mediated cerebrovascular syndrome including embolic stroke, thrombotic stroke or transient ischemic attacks, (c) the treatment or prevention of any thrombotic syndrome occurring in the venous system including deep venous thrombosis or pulmonary embolus occurring either spontaneously or in the setting of malignancy, surgery or trauma, (d) the treatment or prevention of any coagulopathy including disseminated intravascular coagulation (including the setting of septic shock or other infection, surgery, pregnancy, trauma or malignancy and whether associated with multi-organ failure or not), thrombotic thrombocytopenic purpura, thromboanginitis obliterans, or thrombotic disease associated with heparin induced thrombocytopenia, (e) the treatment or prevention of thrombotic complications associated with extracorporeal circulation (e.g. renal dialysis, cardiopulmonary bypass or other oxygenation procedure, plasmapheresis), (f) the treatment or prevention of thrombotic complications associated with instrumentation (e.g. cardiac or other intravascular catheterization, intra-aortic balloon pump, coronary stent or cardiac valve), and (g) those involved with the fitting of prosthetic devices.

Anticoagulant therapy is also useful to prevent coagulation of stored whole blood and to prevent coagulation in other biological samples for testing or storage. Thus the compounds disclosed herein can be added to or contacted with any medium containing or suspected to contain factor IIb/IIIa, and the like, in which it is desired that blood coagulation be inhibited, e.g., when contacting the mammal's blood with material such as vascular grafts, stents, orthopedic prostheses, cardiac stents, valves and prostheses, extra corporeal circulation systems and the like.

Without further description, it is believed that one of ordinary skill in the art can, using the preceding description and the following illustrative examples, make and utilize the disclosed and claimed compounds and practice the disclosed and claimed methods. The following working examples therefore, specifically point out preferred embodiments, and are not to be construed as limiting in any way the remainder of the disclosure.

### EXAMPLES

### Example 1

### Production of ethyl(6-acetamido-chroman-2-yl)acetate Methanol/Toluene Solution

8.80 Kg (34.80 moles) of ethyl (6-aminochroman-2-yl) acetate (described in U.S. patent at the paragraph bridging pages 147 and 48 as a dark oil) was added to 80 L of toluene and the mixture was heated with stirring to 40° C. under reduced pressure to effect dissolution of the oil. The solution was cooled to about −5° C. and 4.40 Kg of pyridine was added. To this solution was added 3.70 Kg of acetyl chloride (47.13 moles) over 1 hour (−5° C.≦T≦5° C.). The reaction mixture was then stirred for at least 30 minutes after which 47 Kg of water was added. The solution was warmed up to room temperature and stirred for at least 15 minutes before decantation. The organic layer was separated and distilled off under reduced pressure (T≦55° C.) until most of the solvent was eliminated. The residue was cooled down to about 15° C., 19.77 Kg of methyl alcohol was added and the distillation was continued until the volume of the residue was about 25 L (ethyl (6-acetamidochroman-2-yl) acetate in a mixture of methyl alcohol and toluene).

¹H-NMR (250 MHz, CDCl₃) 7.34 (d(br), J=2.5 Hz, 1H), 7.13 (s, 1H), 7.05 (dd, J=8.9 Hz, 2.5 Hz, 1H), 6.74 (d, J=8.9 Hz, 1H), 4.46 (qd, J=7.5 Hz, 1.2 Hz, 1H), 4.23 (q, J=7.2 Hz, 2H), 2.84 (ddd, J=16.5 Hz, 5.2 Hz, 4.1 Hz, 1H), 2.60 (dd, J,=15.4 Hz, 6.1 Hz, 1H), 2.6 (s, 3H), 1.2 (m, 1H), 1.77 (m, 1H), 1.31 (t, J=7.2 Hz, 3H)

¹³C-NMR (62.9 MHz, CDCl₃) 170.7, 168.2, 151.4, 130.5, 121.9, 121.8, 119.9, 117.0, 72,.4, 60.7, 40.6, 28.4, 26.9, 24.5, 24.4, 14.2

### Example 2

### Production of (6-acetamido-chroman-2-yl)-acetic Acid

To the 25 L of ethyl (6-acetamido-chroman-2-yl)acetate in a mixture of methyl alcohol and toluene (from Example 1) was added 27.2 Kg of methyl alcohol followed by 57.40 Kg of 1 N aqueous sodium hydroxide (55.19 moles) while maintaining a T≦30° C. The solution was stirred for at least one hour at room temperature. The solvents were distilled off under reduced pressure (at T≦35° C.), until the volume of the residue was about 65 L. The mixture was cooled down to room temperature and 40.65 Kg of toluene was added. The mixture was stirred for about 15 minutes. The organic phase was separated and the aqueous layer was extracted with 19.9 kg of toluene. The pH of the aqueous phase was reduced to 2≦pH≦3 by slow addition of the 25.3 Kg of 2 N aqueous hydrochloric acid. The suspension was stirred for at least 1 hour at room temperature. The crystals were filtered, rinsed with 28 Kg of water and dried under reduced pressure (45° C.≦T≦50° C.) for about 16 hours to yield 7.81 Kg of (6-acetamido-chroman-2-yl)acetic acid (31.33 mole). Yield 90% from combined Examples 1 and 2.

¹H-NMR (250 MHz, DMSO-d6), 12.32 (s(br), 1H), 9.68 (s, 1H), 7.31 (dd, J=2.5 Hz, 1H), 7.18 (dd, J=8.9 Hz, 2.5 Hz, 1H), 6.62 (d, J=8.9 Hz, 1H), 4.31 (qd, J=7.5 Hz, 1.2 Hz, 2H), 2.80 (m, 1H), 2.78 (m, 1H), 2.75 (m, 1H), 2.60 (m, 1H), 1.98 (m, 4H), 1.68 (m, 1H)

¹³C-NMR (62.9 MHz, DMSO-d6), 171.9, 167.7, 150.0, 132.1, 121.6, 120.4, 118.7, 116.2, 72.4, 40.1, 26.5, 24.1, 23.8

### Example 3

### Production of ethyl (6-acetamido-chroman-2-yl)-acetate Methanol/Toluene Solution

660 g (about 2.6 moles) of ethyl (6-amino-chroman-2-yl) acetate (described in U.S. patent at the paragraph bridging pages 147 and 48 as a dark oil) was added to 6 L of toluene and the mixture was heated with stirring to 40° C. under reduced pressure to effect dissolution of the oil. The solution was cooled to about −5° C. in a sodium chloride-ice bath and 293 g of anhydrous pyridine was added in one portion, followed by dropwise addition of 246 g (3.2 moles) of acetyl chloride with good agitation to maintain the temperature in the range of from about −5° C. to about 5° C. the reaction). The reaction mixture was stirred for 30 minutes after addition, after which TLC analysis indicated that the reaction was not yet complete. An additional 50 g (0.6 mole) of pyridine and 50 g (0.45 mole) of acetyl chloride were added and the reaction stirred for 30 more minutes before it was quenched by the addition of 4 L of water. After stirring for 15 minutes, the organic layer was separated. The aqueous layer was extracted with 1 L of toluene and the combined toluene extracts were washed with 2 L of water. After removal of most of the toluene by distillation under reduced pressure (T≦55° C.) until most of the solvent was eliminated. The residue was cooled down to about 15° C., 2.5 L of methyl alcohol was added and the distillation was continued until the volume of the residue was about 1.5 L (ethyl (6-acetamido-chroman-2-yl)-acetate in a mixture of methyl alcohol and toluene).

¹H-NMR (250 MHz, CDCl₃) 7.34 (d(br), J=2.5 Hz, 1H), 7.13 (s, 1H), 7.05 (dd, J=8.9 Hz, 2.5 Hz, 1H), 6.74 (d, J=8.9 Hz, 1H),4.46 (qd, J=7.5 Hz, 1.2 Hz, 1H), 4.23 (q, J=7.2 Hz, 2H), 2.84 (ddd, J=16.5 Hz, 5.2 Hz, 4.1 Hz, 1H), 2.60 (dd, J,=15.4 Hz, 6.1 Hz, 1H), 2.6 (s, 3H), 1.2 (m, 1H), 1.77 (m, 1H), 1.31 (t, J=7.2 Hz, 3H)

¹³C-NMR (62.9 MHz, CDCl₃) 170.7, 168.2, 151.4, 130.5, 121.9, 121.8, 119.9, 117.0, 72,.4, 60.7, 40.6, 28.4, 26.9, 24.5, 24.4, 14.2

### Example 4

### Production of (6-acetamido-chroman-2-yl)-acetic Acid

To the 1.5 L of ethyl (6-acetamido-chroman-2-yl)acetate in a mixture of methyl alcohol and toluene (from Example 5) was added 3.5 L of methyl alcohol followed by 3.5 L of 1 N aqueous sodium hydroxide while maintaining a temperature ≦30° C. The solution was stirred for at least one hour at room temperature. The solvents were distilled off under reduced pressure (at T≦35° C.), until the volume of the residue was about 5 L. The mixture was cooled down to room temperature and 6 L of toluene was added. The mixture was stirred for about 15 minutes. The organic phase was separated and the aqueous layer was extracted with 2×2 L of toluene. The pH of the aqueous phase was reduced to 2≦pH≦3 by slow addition of about 2.75 L of 2 N aqueous hydrochloric acid. The suspension was stirred for at least 1 hour at room temperature. The crystals were filtered, rinsed with 10 L of water and dried under reduced pressure (45° C.≦T≦50° C.) for about 16 hours to yield 598 g of 6-acetamidochroman-2-yl acetic acid (2.4 mole). Yield from combined Examples 3 and 4=92%.

¹H-NMR (250 MHz, DMSO-d6), 12.32 (s(br), 1H), 9.68 (s, 1H), 7.31 (dd, J=2.5 Hz, 1H), 7.18 (dd, J=8.9 Hz, 2.5 Hz, 1H), 6.62 (d, J=8.9 Hz, 1H), 4.31 (qd, J=7.5 Hz, 1.2 Hz, 2H), 2.80 (m, 1H), 2.78 (m, 1H), 2.75 (m, 1H), 2.60 (m, 1H), 1.98 (m, 4H), 1.68 (m, 1H)

¹³C-NMR (62.9 MHz, DMSO-d6), 171.9, 167.7, 150.0, 132.1, 121.6, 120.4, 118.7, 116.2, 72.4, 40.1, 26.5, 24.1, 23.8

### Example 5

### Production of D-alaninol Salt of (6-acetamido-chroman-2-yl)-acetic Acid

477.42 g (1.92 mole) of racemic (6-acetamido-chroman-2-yl)-acetic acid (from Example 4 or 6, above was suspended in 2.86 L of methanol (1 g/6 mL) at 20° C. During addition of D-alaninol (80.53 g=0.55 equivalents with respect to the chroman racemate) the whole mixture dissolved completely. This solution was heated to reflux. Crystallization may start at reflux, but the solution was maintained at reflux for about 45 minutes and then the temperature was decreased gradually to 20° C. over a period of 60 minutes. The crystalline suspension was then stirred for an additional 3.5 hours. After filtration, the crystals were washed with 150 mL of isopropanol, dried overnight at 45° C. under reduced pressure to yield 260 g of enriched diastereomeric n-salt (D-alaninol N-salt of (6-acetamido-chroman-2-yl)-acetic acid). Yield for crude S enantiomer salt=42%. The optical rotation at 20° C.=+64.0° (c=2, H₂O): calculated composition 95.1/4.9 (S>R)=90.2% ee. Chiral HPLC: composition 95/5 (S>R)=90% ee (enantiomeric excess).

To purify the enriched S enantiomer even further the enriched diasteromer (about 90% ee) was suspended in 4.8 L of methanol. The mixture was heated at reflux for 16 hours. The suspension was cooled down to room temperature and stirred for 1 hour. After filtration, the crystals were washed with 480 mL of methyl alcohol and dried overnight at 50° C. under reduced pressure to give 211 g of optically pure n-salt. Yield for this step was 82.1% with an overall resolution yield and purification of 34.5%. (Assuming a 50/50 mixture of the chromane racemate, this was a 69% isolation yield of the amount of (S) enantiomer which was present in the racemate.) The optical rotation at 20° C.=+71.3° (c=2, H₂O); calculated composition 99.7/0.3 (S>R)=99.4% ee, and the (R) enantiomer was no longer detectable by chiral HPLC.

### Example 6

### Larger-Scale Production of D-alaninol Salt of (6-acetamido-chroman-2-yl)-acetic Acid

7.71 Kg (30.91 mole) of racemic (6-acetamido-chroman-2-yl)-acetic acid (from Example 5, above) was processed essentially as set forth in Example 7, except that in the purification step the solution was only heated to reflux and maintained with stirring for 8 hours to provide 3.41 Kg of optically pure n-salt (10.5 moles). The overall yield for the production of the crude and then further purified (S) enantiomer was 34%. The optical rotation at 20° C.=+71.75° (c=2, H₂O); and chiral HPLC shows a composition of 98.95/1.85 (S>R)=97.9% ee.

mp 218.4° C. (capillary)

¹H-NMR (400 MHz, DMSO-d6), 9.75 (s, 1H), 7.33 (d, J=2.5 Hz, 1H), 7.19 (dd, J=8.9 Hz, 2.5 Hz, 1H), 6.62 (d, J=8.9 Hz, 1H), 4.29 (qd, J=7.5 Hz, 1.2 Hz, 1H), 3.25 9dd, J=11.3 Hz, 4.3 Hz, 1H), 3.39 (dd, J=11.3 Hz, 6.7 Hz, 1H), 3.15 (m, 1H), 2.80 (ddd, J=16.5 Hz, 10.4 Hz, 5.2 Hz, 1H), 266 (dm, J=16.5 Hz, 1H), 2.53 (m, 2H), 1.98 (m, 4H), 1.64 (m, 1H), 1.13 (d, J=6.6 Hz, 3H)

¹³C-NMR (100 MHz, DMSO-d6), 173.3, 167.6, 150.4, 131.7, 121.6, 120.3, 118.6, 116.1, 73.7, 64.6, 48.4, 40.1, 26.8, 24.4, 23.8, 16.8

IR (KBr) 3316, 3029, 2974, 2925, 1570, 1494, 1408 cm⁻¹

### Example 7-10

### Production of L-alaninol Salt of 2-(6-acetamido-chroman-2-yl)acetic Acid and Isolation of Enriched L-alaninol Salt of (R) Enantiomer.

Examples 3-6 are repeated with essentially the same results except that L-alaninol is reacted with the racemic chromane acetic acid ester and the (2R) enantiomer was obtained in enriched form with essentially the same yields as the (2S) enantiomer in Examples 3-6.

### Example 11

### Production of ethyl 2-(2S)-(6-amino-chroman-2-yl)-acetate Hydrochloride Salt

257 g of the (S) enantiomer enriched as set forth in Examples 5-6 (about 92% ee with respect to the (S) enantiomer) was refluxed for 16 h under nitrogen in 2.7 L of 3N sulfuric acid solution in absolute ethyl alcohol. The reaction mixture was then concentrated under reduced pressure (rotatory evaporator) to a whole mass of 1.03 Kg. Then 5 L of toluene and 430 g of sodium hydrogen carbonate in 1 L of water were added successively (at neutralization the reaction mixture becomes pink). After 10 minutes under stirring, the toluene was separated from the aqueous layer. The aqueous layer was then extracted successively 4 times with 1 L of toluene until completion of extraction (monitoring by HPLC to follow extraction process). The pooled toluenic phase was dried on magnesium sulfate and after filtration concentrated to 4 L. 430 mL of a hydrochloric acid 3.6 N ethereal solution was then added to precipitate the crude hydrochloride salt. After 1 hour stirring at 20° C. the hydrochloride salt was filtrated and rinsed with 500 mL of toluene. This material was dried under reduced pressure at 45° C. to give 199 g of crude ethyl (2S)-(6-amino-chroman-2-yl) acetate. Yield 92%. Optical rotation at 20° C.=+90.30 (c=0.2, EtOH); estimated composition 96/4 (S>R)=92% ee.

This crude ester was further purified by suspending the 199 g of crude product in 980 mL ethanol (ratio of about 1 g/4.5 mL of ethanol) and kept at reflux for 1 hour (digestion). The suspension was cooled to 20° C. and stirred for an additional 4 hours. After filtration and washing with a little volume of ethyl alcohol, the white crystals were dried for 24 hours at 42° C. under reduced pressure to yield 177 g of purified ethyl (2S)-(6-amino-chroman-2-yl) acetate. Yield 88.5% from the crude ester and overall 81.4% from the amino hydrochloride salt starting material. Optical rotation 20° C.=+94.6° (c=0.2, EtOH); estimated composition 98.5/1.5 (S>R)=97% ee.

### Example 12

### Larger-Scale Production of Ethyl 2-(2S)-(6-amino-chroman-2-yl) Acetate Hydrochloride Salt

2.88 Kg (8.87 moles) of the (S) enantiomer enriched as set forth in Examples 5-6 (about 98% ee with respect to the (S) enantiomer) was added to 21 Kg of absolute ethyl alcohol under stirring at room temperature. 2.42 L of concentrated sulfuric acid were slowly added to the solution and the mixture was refluxed under stirring for at least 16 h under nitrogen. The solution was cooled to room temperature and slowly added to a mixture of 26.6 L of 10% aqueous sodium hydrogen carbonate and 29 L of dichloromethane while maintaining the temperature at about 5° C. The stirring was continued for at least 15 minutes at the same temperature before decantation (pH of the solution was over 7). The organic layer was separated and washed again with 36.7 L of 2% aqueous sodium hydrogen carbonate while maintaining the temperature at about 5° C.

The organic phase was concentrated to minimum stirrable volume under reduced pressure while keeping the temperature below 40° C. 29 L of toluene was added under vacuum and distillation was continued until the volume was about 12 L, while maintaining the temperature below 50° C. The reaction mixture was then cooled to about 19° C. and 1.45 L of 6.2 M hydrochloric acid in ethyl alcohol was slowly added in order to maintain the temperature between 10 and 20° C. The crystals were maturated under stirring for at least 16 h at the same temperature, filtered and washed with 6 L of toluene. The product was dried for at least 16 hours under reduced pressure while maintaining the temperature between 45 and 50° C. to afford 2.16 Kg (7.96 mole) of ethyl (2S)-(6-amino-chroman-2-yl) acetate hydrochloride. Yield 89.85 based on the starting material. Optical rotation at 20° C.=+98.0° (c=0.2, EtOH); estimated composition 99/1 (S>R)=98% ee.

¹H-NMR (250 MHz, DMSO-d6), 9.97 (s(br), 3H), 7.05 (m, 2H), 6.80 (m, 1H), 4.42 (qd, J=75 Hz, 1.2 Hz, 1H), 4.14 (q, J=6.7 Hz, 2H), 2.88 (ddd, J=16.5 Hz, 10.4 Hz, 5.2 Hz, 1H), 2.8 (dd, J=11.3 Hz, 4.3 Hz, 1H), 2.76 (m, 1H), 2.74 (dd, J=11.3 Hz, 6.7 Hz, 1H), 2.05 (m, 1H), 1.70 (m, 4H), 1.22 (t, J=6.7 Hz, 3H)

¹³C-NMR (62.9 MHz, DMSO-d6), 170.2, 153.4, 123.9, 123.1, 121.9, 117.3, 72.6, 60.1, 39.7, 25.9, 23.7, 14.1

IR (KBr) 2907, 2632, 1733, 1504, 1201 cm⁻¹

### Example 13

### Production of Racemic (6-acetamido-chroman-2-yl)acetic Acid from an Enriched Methanol Solution of (R>S)(6-acetamido-chroman-2-yl)acetic Acid

(a) Production of (R>S) methyl (6-acetamido-chroman-2-yl)acetate

A fraction of 843.6 g of the mother liquors (methanol solution) of the resolution of the (2S) enantiomer from Example 5, containing about (0.288 mole) of the enriched (R>S) enantiomer is concentrated under reduced pressure to 310 g. To this concentrated solution at −25° C. is added 30 mL of thionyl chloride (0.411 moles=1.43 equiv.) dropwise over 10 minutes. The esterification of the (R>S) of the (6-acetamido-chroman-2-yl)acetic acid with the methanol of the solvent is completed by agitation at room temperature for about 40 minutes. The mixture is neutralized (pH 7-8) at 0° C. by addition of 81.3 mL 25% aqueous ammonia. 170 mL of water is then added to dissolve the precipitated ammonium chloride. The aqueous phase is extracted successively by 500 mL and 250 mL of ethyl acetate. After drying on magnesium sulfate, the pooled organic phase is concentrated and the solid residue is dried under reduced pressure to give 70.1 g of crude (R>S) methyl (6-acetamido-chroman-2-yl)acetate. Yield=92.2%.

¹H-NMR (250 MHz, CDCl₃) 7.29 (s(br), 1H), 7.28 (d, J=2.4 Hz, 1H), 7.03 (dd, J=8.7 Hz, 2.4 Hz, 1H), 6.38 (d, J=8.7 Hz, 1H), 4.42 (qd, J=8.0 Hz, 2.1 Hz, 1H), 3.73 (s, 3H), 2.82 (ddd, J=16.7 Hz, 10.4 Hz, 6.0 Hz, 1H), 2.77 (dd, J,=15.4 Hz, 7.2 Hz, 1H), 2.69 (dt, J=15.6 Hz, 4.4 Hz, 1H), 2.59 (dd, J=15.4 Hz, 5.9 Hz, 1H), 2.11 (s, 3H), 2.05 (m, 1H), 1.74 (m, 1H)

¹³C-NMR (100 MHz, CDCl₃) 171.2, 168.4, 151.2, 130.7, 121.8, 121.7, 120.0, 116.7, 72.3, 51.8, 40.3, 27.0, 24.4, 24.2

(b) Production of (R=S) methyl (6-acetamido-chroman-2-yl)acetate

70.1 g of crude methyl ester from (a) is dissolved in 500 mL of methyl alcohol. To this solution is added a solution of potassium methoxide prepared from 26.3 g of potassium tert-butoxide and 300 mL of methyl alcohol. The reaction mixture is stirred 50 minutes at 0° C. and 43.5 hours at room temperature to complete racemization until an approximately S=R racemate is obtained. (The physical and chemical data of the product is essentially the same as that obtained from directly forming a methyl ester from the (6-acetamido-chroman-2-yl)acetic acid produced in Example 4 prior to enantiomeric resolution.)

(c) Production of (R=S) (6-acetamido-chroman-2-yl)acetic Acid

The reaction mixture of (b) is saponified to remove the methyl ester and obtain the sodium salt of the free acid at room temperature by adding and excess of a base (about 145 mL) of 1 N aqueous sodium hydroxide sufficient to raise the pH of the mixture to a pH between about 11-12. After 2 hours stirring at room temperature, the methyl alcohol in the solution is evaporated under reduced pressure. The sodium salt is then precipitated from the aqueous solution by adding a sufficient amount of 1 N aqueous hydrochloric acid to reduce the pH to about 1 to 2 (about 250-400 mL). The white crystals are filtered and dried under pressure to give 53.8 g of racemic (6-acetamido-chroman-2-yl)acetic acid having essentially the same physical and chemical properties as the racemate produced in Example 4, above.

### Example 14

### Production of Racemic (6-acetamido-chroman-2-yl)acetic Acid from Enriched Methanol Solution of (R>S) (6-acetamido-chroman-2-yl)acetic Acid

(a) Separation of (R>S) (6-acetamidochroman-2-yl)acetic Acid from Enriched Methanol Solution of (R>S) (6-acetamido-chroman-2-yl)acetic Acid

The R>S (6-acetamido-chroman-2-yl)acetic acid in methanol of Example 6 (mother liquor) is treated with a sufficient amount of 1 N aqueous sodium hydroxide (about 25 moles) to form a sodium salt while maintaining a T≦30° C. The solution is stirred for at least one hour at room temperature. The solvents are distilled off under reduced pressure (at T≦35° C.), until the volume of the residue is reduced to about 25 L. The mixture is cooled down to room temperature and 30 L of toluene is added. The mixture is stirred for about 15 minutes. The organic phase is separated and the aqueous layer is extracted with 2×10 L of toluene. The pH of the aqueous phase is reduced to 2≦pH≦3 by slow addition of the 14 L of 2N aqueous hydrochloric acid. The suspension is stirred for at least 1 hour at room temperature. The crystals are filtered, rinsed with 50 L of water and dried under reduced pressure (45° C.≦T≦50° C.) for about 16 hours to yield 4.5 Kg of (6-acetamido-chroman-2-yl)acetic acid (18.04 mole).

(b) Formation of (R>S) ethyl (6-acetamido-chroman-2-yl)acetate from Crude (R>S) (6-acetamido-chroman-2-yl)acetic Acid

The crude (R>S) product of (a) 4.5 Kg (18.04 moles) was added to 8 L of ethanol. To this solution at −25° C. is added slowly over ½hour 640 mL of thionyl chloride (25.6 moles=1.43 equiv.). The esterification of the (R>S) of the (6-acetamido-chroman-2-yl)acetic acid with the ethanol is completed by agitation at room temperature for about 40 minutes. The mixture is neutralized (pH 7-8) at 0° C. by addition of 5.1 L of aqueous ammonia 25% and 11 L of water is then added to dissolve the precipitated ammonium chloride. The aqueous phase is extracted successively by 32 L and 16 L of ethyl acetate. After drying on magnesium sulfate, the pooled organic phase is concentrated and the solid residue is dried under reduced pressure to give 4.93 K g of crude (R>S) ethyl (6-acetamido-chroman-2-yl)acetate. Yield=93.3%.

(c) Production of (R=S) ethyl (6-acetamido-chroman-2-yl)acetate

In a 22-L 3-necked RB flask equipped with a heating mantle, condenser, overhead mechanical stirrer and thermocouple was charged 10 L of absolute ethanol, 4.93 Kg (16.83 mole) of R>S ethyl (6-acetamido-chroman-2-yl)acetate from (b) and 320 mL (751 mmoles) of sodium ethoxide, 21% w/w in ethanol. The mixture was heated to 45° C. for 6 hours. Analysis of the mixture by rotation indicates complete reaction as compared to data from an ethyl ester purified from the racemate of Example 3. To the mixture was then added 60 mL (1.01 moles) of acetic acid and the mixture was allowed to cool to room temperature.

(d) Production of (R=S) (6-acetamido-chroman-2-yl)acetic Acid

The reaction mixture of (c) is saponified to remove the methyl ester and obtain the sodium salt of the free acid at room temperature by adding an excess of 1 N aqueous sodium hydroxide base sufficient to raise the pH of the mixture to a pH between about 11-12. After 2 hours stirring at room temperature, the ethyl alcohol of the solution is then evaporated of under reduced pressure. The sodium salt is then precipitated from the basic aqueous solution by adding a sufficient amount of 1 N aqueous hydrochloric acid to reduce the pH to about 1 to 2. The white crystals are filtered and dried under pressure to give 4.1 Kg (97.1% yield for steps (c) and (d), 90.5% yield for steps (b), (c) and (d) of racemic (6-acetamido-chroman-2-yl)acetic acid having essentially the same physical and chemical properties as the racemate produced in Example 4, above.

¹H-NMR (250 MHz, DMSO-d6), 12.32 (s(br), 1H), 9.68 (s, 1H), 7.31 (dd, J=2.5 Hz, 1H), 7.18 (dd, J=8.9 Hz, 2.5 Hz, 1H), 6.62 (d, J=8.9 Hz, 1H), 4.31 (qd, J=7.5 Hz, 1.2 Hz, 2H), 2.80 (m, 1H), 2.78 (m, 1H), 2.75 (m, 1H), 2.60 (m, 1H), 1.98 (m, 4H), 1.68 (m, 1H)

¹³C-NMR (62.9 MHz, DMSO-d6); 171.9, 167.7, 150.0, 132.1, 121.6, 120.4, 118.7, 116.2, 72.4, 40.1, 26.5, 24.1, 23.8

In view of the above description it is believed that one of ordinary skill can practice the invention. The examples given above are non-limiting in that one of ordinary skill in view of the above will readily envision other obvious permutations and variations without departing from the principal concepts embodied therein. Such permutations and variations are also within the scope of the disclosure.

What is claimed is:

1. A process for making enantiomerically enriched 2-[(S>R) 6-aminochroman-2-yl] acetic acid, comprising:
(a) protecting the amino group by conversion to an acetamido group as follows: 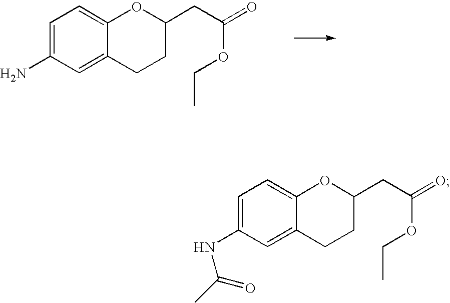
(b) converting the ester to the free acid;
(c) reacting the free acid with a slight excess of D-alaninol in alcholic solution, and separating a first quantity of diastereomeric salt as follows: 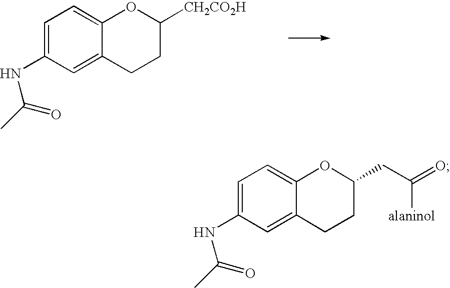
(d) adding thionyl chloride to the (R>S) mixture in the mother liquor containing ROH to esterify the acid followed by neutralization as follows: 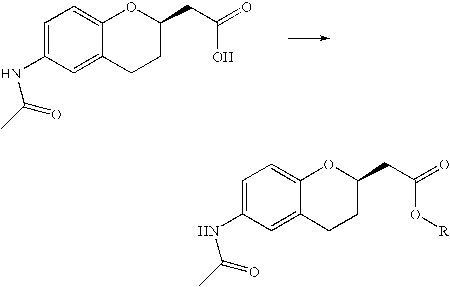
where R is C₁-C₆ alkyl;
(e) racemizing the compound from the mother liquor at the 2-position by opening and closing the pyran ring by the addition of a base: 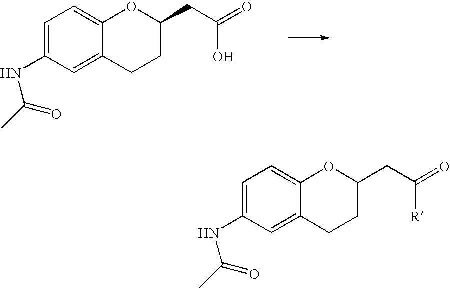
where R′ is H or C₁-C₆ alkyl;
(f) repeating the procedure of (c), either directly of after repeating the procedure of (b), with the solution obtained in (e) to obtain a second quantity of diaster omeric salt, wherein the procedure of (b) is repeated if R′ is not H;
(g) heating the first and second quantities of diastereomeric salt in a solvent comprising ROH in the presence of acid followed by neutralization as follows:

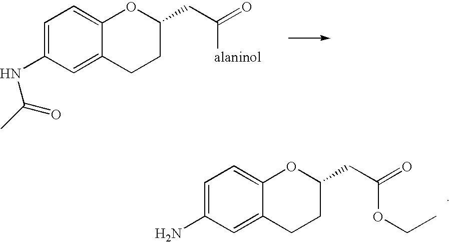

2. The process of claim 1, further comprising:
(g) addition of an acid halide to an organic solution of the product of (f) followed by recovery of the precipitated amino halide salt.

3. A process for making enantiomerically enriched 2-[(S>R) 6-aminochroman-2-yl] acetic acid, comprising:
(a) protecting the amino group by conversion to an acetamido group as follows: 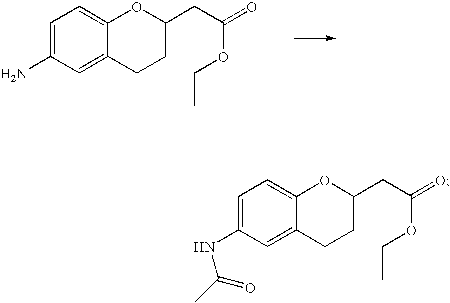
(b) converting the ester to the free acid;
(c) reacting the free acid with a slight excess of D-alaninol in alcholic solution, and separating a first quantity of diastereomeric salt as follows: 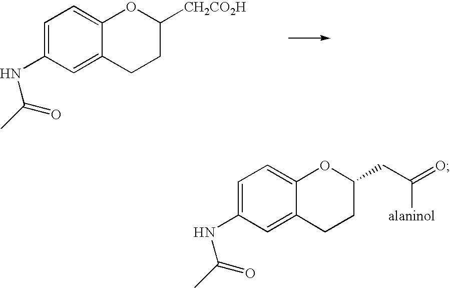
(d) adding potassium or sodium hydroxide to the (R)>(S) mother liquor, followed by acid to precipitate the 2-[6-acetamido-chroman-2-yl] acetic acid;
(e) dissolving the product from (d) in solvent comprising ROH and adding thionyl chloride to esterify the acid as follows: 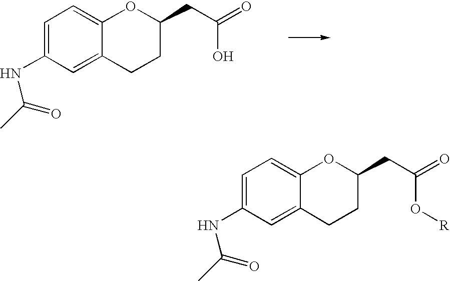
where R is C₁-C₆ alkyl;
(f) racemizing the compound in the mother liquor at the 2-position by opening and closing the pyran ring by the addition of a base: 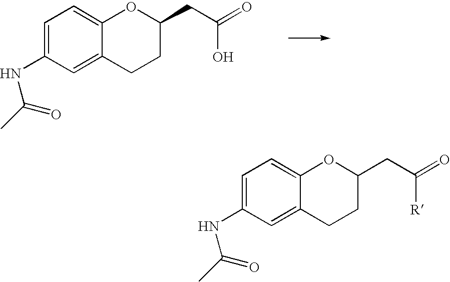
where R′ is H or C₁-C₆ alkyl;
(g) repeating the procedure of (c), either directly of after repeating the procedure of (b), with the solution obtained in (f) to obtain a second quantity of diastereomeric salt, wherein the procedure of (b) is repeated if R′ is not H;
(h) heating the first and second quantities of diastereomeric salt in a solvent comprising ROH in the presence of acid followed by neutralization as follows:

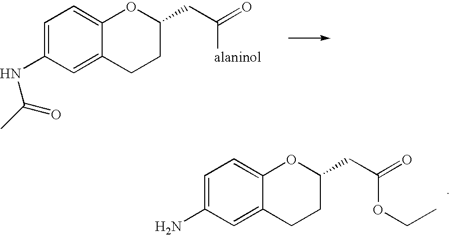

4. The process of claim 1, further comprising:
(g) addition of an acid halide to an organic solution of the product of (f) followed by recovery of the precipitated amino halide salt.
